(19) United States
(12) Patent Application Publication
(10) Pub. No.: US 2025/0169751 A1
(43) Pub. Date: May 29, 2025

(54) PERIPHERAL ARTERY DISEASE SCREENING

(71) Applicant: Kent Imaging Inc., Calgary (CA)

(72) Inventors: Charles A. Andersen, Tacoma, WA (US); Lee Rogers, Calgary (CA)

(21) Appl. No.: 18/961,273

(22) Filed: Nov. 26, 2024

Related U.S. Application Data
(60) Provisional application No. 63/603,113, filed on Nov. 27, 2023.

(51) Int. Cl. A61B 5/00; G06T 7/00; G16H 50/70

(52) U.S. Cl. CPC A61B 5/4848; A61B 5/0075; A61B 5/0082; A61B 5/4842; A61B 5/7271; G06T 7/0016; G16H 50/70; G06T 2207/10036; G06T 2207/30104

(57) ABSTRACT

Disclosed are methods and systems for determining an extent of PAD. The method may include a step of comparing a pre-PAD treatment oxygenation readings by obtaining a supine reading and limb raise reading to post-PAD treatment oxygenation readings of the limb or the portion thereof using a non-contact NIRS camera of a limb or a portion thereof, distal to a PAD treatment site using a supine reading and limb raise reading to post-PAD treatment oxygenation readings of the limb or the portion thereof using a second supine reading and a second limb raise reading. An indication may be presented of an improvement or a non-improvement in PAD in the limb based on a difference in oxygenation from pre-PAD treatment readings to post-PAD treatment oxygenation readings of the limb.

### CROSS REFERENCE TO RELATED APPLICATION

This application claims the benefit of U.S. Provisional Application 63/603,113 filed on Nov. 27, 2023, the entire contents of which are incorporated herein.

### BACKGROUND

In recent years multiple systematic reviews have been published focusing on the utility of near-infrared spectroscopy (NIRS) for the assessment, screening, and/or diagnosis of peripheral artery disease (PAD). PAD is commonly characterized by the narrowing or blocking of the blood vessels that carry blood away from the heart to the peripheral limbs (e.g., legs, lower extremities, arms, upper extremities, etc.). A unifying theme for the utilization of NIRS to diagnose PAD has been that the current evidence is lacking, suggesting “the clinical relevance of routine use of NIRS to diagnose PAD is unproven” or that, “evidence seems too low to define this technique as a gold standard.” However, this lack of evidence appears to come from a lack of standardization in metrics, protocols, and devices for assessing PAD.

The ankle-brachial index (ABI) is the most common diagnostic test used to evaluate the presence of PAD. However, ABIs can be falsely elevated because of medial arterial calcinosis and do not measure tissue oxygenation of the foot. Certain applications of NIRS are capable of measuring superficial tissue oxygenation. Moreover, NIRS technology is promising as a quick, non-contact, and non-invasive vascular assessment tool. However, currently there is little evidence for how to interpret the images as a vascular assessment.

### SUMMARY

It would be helpful to have a technique that can diagnose, screen, and assess peripheral artery disease utilizing NIRS as well as having guidelines for interpretation of the NIRS readings. In one aspect, a method for determining effectiveness of peripheral artery disease (PAD) treatment is provided. The method may include a step of using a non-contact NIRS camera to image a limb, or portion thereof, to compare oxygenation readings from a pre-PAD treatment timepoint using a supine reading and limb raise reading to oxygenation readings of a limb, or portion thereof, from a post-PAD treatment timepoint using a second supine reading and a second limb raise reading. For the limb-raised pre-PAD treatment oxygenation reading, the limb is raised to between 20 and 90 degrees, preferably about 45 degrees, from supine for 30 seconds to 5 minutes, preferably about 1 minute. For the limb-raised post-PAD treatment oxygenation reading, the limb is raised to approximately the same angle (within 5-10 degrees) as the pre-PAD treatment oxygenation reading taken for approximately the same time (within about 5 to 30 seconds) as the pre-PAD treatment oxygenation readings. An indication may be presented of an improvement or a non-improvement in PAD in the limb based on a difference in tissue oxygenation from the supine to raised position from the pre-PAD treatment readings to post-PAD treatment oxygenation readings of the limb. In some embodiments the methods, system and devices herein diagnose PAD. In some embodiments the methods, system and devices herein screen for severity of PAD. In some embodiments the methods, system and devices herein assess the improvement in PAD following a treatment thereof.

In another aspect, a method for predicting the need for peripheral artery disease (PAD) treatment in an individual is provided. A supine oxygenation reading of a limb or a portion thereof and a raised oxygenation reading of the limb or the portion thereof whereby the limb is elevated to between 20 and 90 degrees, preferably about 45 degrees, from supine for 30 seconds to 5 minutes, preferably about 1 minute, are acquired using a non-contact NIRS camera. The method includes comparing the difference in the supine reading to the raised limb reading to expected differences between these readings as compared to a past difference in the same limb or compared to a normal difference in the readings based on normative population values from healthy individuals without PAD, and indicating a need in the individual for PAD treatment or diagnosis based on the difference comparison.

In a further aspect, another method for predicting need for peripheral artery disease (PAD) treatment in an individual is provided. In response to ABI or (toe-brachial index) TBI testing being unavailable due to calcification in the limb or not interpreted as being predictive of PAD, using a non-contact NIRS camera to take a supine oxygenation reading of a limb or a portion thereof and a raised-limb oxygenation reading of the limb or a portion thereof by raising the limb to between 20 and 90 degrees, preferably about 45 degrees, from supine for 30 seconds to 5 minutes, preferably about 1 minute. The method further includes comparing the difference in the supine oxygenation reading to the raised-limb oxygenation reading to expected differences between these readings as compared to a past difference in the same limb or compared to a normal difference in the readings based on normative population values from healthy individuals without PAD, and indicating a need in the individual for PAD treatment based on the difference comparison.

In yet a further aspect, a system determining the effectiveness of peripheral artery disease (PAD) treatment is provided. The system may include a non-contact NIRS camera and a module. The module may be configured to compare a first oxygenation reading of a limb or a portion thereof of an individual who has received a PAD treatment to a second oxygenation reading of the limb or the portion thereof. The first readings are taken prior to a PAD treatment and the second readings are taken following the PAD treatment, and readings are taken distal to a site of the PAD treatment and where both readings are taken in a supine position and raised-limb position. The first readings indicate a first difference in oxygenation of the limb or the portion thereof in a supine position and the limb or the portion thereof in a raised-limb position. The raised-limb position is a position where the limb is raised to between 20 and 90 degrees, preferably about 45 degrees, from supine for 30 seconds to 5 minutes, preferably about 1 minute. The second readings indicate a second difference following the PAD treatment in oxygenation of the limb or a portion thereof in a supine position and the limb or the portion thereof in a raised-limb position where the limb is raised to between approximately 20 and 90 degrees, preferably about 45 degrees, from supine for approximately 30 seconds to 5 minutes, preferably about 1 minute, and the second reading is after the PAD treatment or after a time longer than one week, or the first reading is taken of the limb or the portion thereof in a supine position, and the second reading is taken of the limb or the portion thereof in a raised-limb position. The time longer than one week may be a time since the pre-PAD assessment, the actual procedure that did the treatment, the last clinic visit, or the like. The module is further configured to optionally provide a prediction or flag for potential diagnosis of PAD disease, an improvement in oxygenation of the limb or the portion thereof, or an indication of non-improvement of the individual's PAD disease. The NIRS camera and the module may be configured in a single device or are in separate devices.

In another additional aspect, the system is operable to capture a first imaging sequence, captured prior to PAD treatment which includes a supine image and raised-limb image. Capture a second imaging sequence, captured after PAD treatment which again includes a supine image and raised-limb image. The system compares the first imaging sequence to the second imaging sequence and determines an effectiveness of the PAD treatment. The raised-limb image may be captured when the limb is raised to between 20 and 90 degrees, preferably about 45 degrees, from supine for 30 seconds to 5 minutes, preferably about 1 minute.

### BRIEF DESCRIPTION OF THE DRAWINGS

To easily identify the discussion of any particular element or act, the most significant digit or digits in a reference number refer to the figure number in which that element is first introduced. The patent or application file contains as least one drawing executed in color. Copies of this patent or patent application publication with color drawing(s) will be provided by the Office upon request and payment of the necessary fee.

FIG. 1 illustrates an aspect of the subject matter in accordance with one embodiment.

FIG. 2 illustrates another aspect of the subject matter in accordance with one embodiment.

FIG. 3 illustrates an exemplary method for determining effectiveness of peripheral artery disease (PAD) treatment in accordance with one embodiment.

FIG. 4 illustrates an example of a method for predicting need for peripheral artery disease (PAD) treatment in an individual in accordance with one embodiment.

FIG. 5 illustrates another exemplary method for predicting need for peripheral artery disease (PAD) treatment in an individual in accordance with one embodiment.

FIG. 6 illustrates an example block diagram of an exemplary system for implementing aspects of the disclosed subject matter.

### DETAILED DESCRIPTION

It would be helpful to have a technique that can diagnose, screen, and assess PAD utilizing NIRS as well as having guidelines for interpretation of the NIRS readings. Disclosed herein, in some respects, are systems and methods for diagnosing and/or assessing peripheral artery disease (“PAD”) for a body part (e.g., limb, such as leg, arm, etc.) of a subject. Systems and methods herein may include obtaining near-infrared spectroscopy (NIRS) images of the limb for the subject. As used herein, the term “subject” may be interchangeably used with patient, person, or individual. Systems and methods may include obtaining and comparing NIRS images of the limb in a supine position and in a raised position (e.g., a leg raised position). The NIRS images may also be compared with images from a cohort of individuals that are not suffering from PAD and/or are suffering from PAD, so as to determine a diagnosis and/or assessment of PAD on the subject.

Disclosed herein in other aspects are systems and methods for assessing a treatment of PAD treatment oxygenation. Systems and methods may include a step of comparing pre-PAD treatment oxygenation readings by obtaining a supine reading and limb raise reading to post-PAD treatment oxygenation readings of the limb or the portion the limb using a non-contact NIRS camera. A supine image of a limb or a portion thereof, which may be distal to a proposed PAD treatment site, may be obtained while the limb is supine. Prior to PAD treatment, the pre-PAD treatment supine image and a pre-PAD raised limb image is obtained by the non-contact NIRS camera. After PAD treatment, the non-contact NIRS camera may capture a supine limb image and a limb raise image. The system may be operable to determine post-PAD treatment oxygenation readings of the limb or the portion thereof using a second supine image and a second limb raise image. An indication may be presented of an improvement or a non-improvement in PAD in the limb based on a difference in oxygenation from pre-PAD treatment readings to post-PAD treatment oxygenation readings of the limb.

Successfully evaluating NIRS images for critical limb ischemia (PAD), which is a severe stage of PAD, was not possible using a single NIRS supine image. Baseline images show no evidence of ischemia in the oxygenation images of subjects with severe PAD likely due to stasis. However, if there is a suspicion of possible PAD based on family history, other signs, or symptoms, then using a provocative maneuver, such as single leg elevation maneuver, in combination with NIRS imaging may be an effective way to either detect PAD or direct attention to other causes of reduced oxygenation or de-oxygenation. In some cases, where an initial NIRS image appears normal, patient history of PAD, or other signs and/or systems of PAD, a provocative maneuver (e.g., a leg elevation) may be used in the interpretation the NIRS data. In addition, NIRS imaging in conjunction with a provocative maneuver may provide a way to evaluate PAD in patients that could not be assessed through standard techniques (e.g., ankle brachial index or “ABI”) due to conditions, such as calcification.

Exemplary treatment methods for peripheral artery disease (PAD) focuses on reducing symptoms and preventing further progression of the condition. In most cases, lifestyle changes, exercise, claudication medications, or the like may help to slow the progression, or even reverse the symptoms, of PAD.

In some aspects, disclosed herein, system and methods for diagnosing PAD and/or assessing a severity and/or effectiveness of a treatment for PAD is based on determinations, such as 1) supine resting tissue oxygen saturation (StO₂) values were not different among cohorts with no PAD and in groups of varying PAD severity; and 2) a 60 second leg elevation maneuver was able to identify patients with severe PAD, but not mild or moderate PAD. Further, NIRS imaging was used in some cases, both before and after revascularization of the lower limb, and it was a precise determiner of post-surgical outcomes. Based upon a single imaging timepoint in a dependent position, NIRS imaging as a standalone modality may not accurately screen for PAD. However, NIRS imaging used in conjunction with a provocative maneuver may serve as a fast, precise, and accurate tool to diagnose severe PAD and it may have utility in assessing post-surgical outcomes.

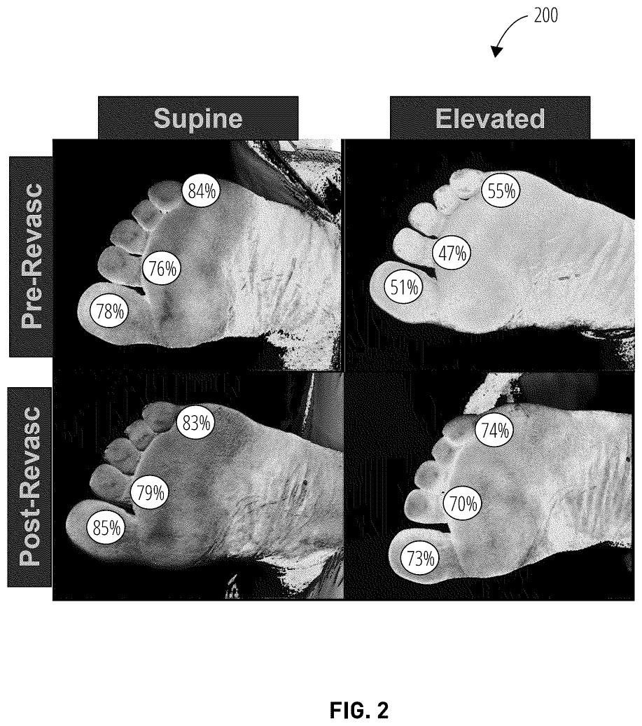

For example, there is no significant difference in StO₂ between the normal patients and the severe PAD patients when imaged at a supine rest condition (FIG. 2). This adds to a dichotomy of literature where some studies have found significantly lower StO₂ or oxyhemoglobin (HbO) (i.e., normal, oxygen-carrying hemoglobin) in PAD participants compared to healthy control participants, while other work has found no difference between PAD and controls. While research supports the latter, this discrepancy highlights the historical ineffectiveness of only interpreting NIRS data from a single baseline timepoint. In this disclosure, the NIRS images have higher clinical relevance when assessing change over time or with a provocative maneuver. Imaging patients only in a dependent position (e.g., supine, sitting), where gravity can influence limb perfusion, is not only inconsistent in identifying disease but may also give false assurance of sufficient arterial flow.

Alternatively, the body of literature with provocative maneuvers (e.g., exercise, post occlusive reactive hyperemia) combined with NIRS imaging/readings have provided more promising results. Multiple studies have found that changes in (i.e., delta) tissue oxygen saturation (ΔStO₂), oxyhemoglobin (ΔHbO), and/or hemoglobin (ΔHb) were significantly different between PAD and controls with an exercise regimen or with post-occlusive reactive hyperemia (PORH) protocols. However, an exercise regimen can be time consuming and may not be followed. Alternatively, the PORH protocols may utilize occlusion cuffs as well as heavy and unwieldy blood perfusion imagers, such as the Perimed Pericam PSI NR, or the like.

Thus, the utility of NIRS imaging/readings as disclosed herein lies in a response elicited by a perturbation and indicates the need to assess NIRS imaging/readings during a provocative maneuver to ensure an accurate diagnosis of severe PAD without having to adhere to an exercise regimen and/or the use of unwieldy imaging equipment, occlusion cuffs, or the like.

The leg raise protocol while utilizing NIRS imaging/reading has multiple advantages over exercise/PORH maneuvers and other routine PAD assessments. First, a recent systematic review on tissue perfusion tests to diagnose PAD called for non-invasive, contact-free, and quick diagnostic tests that could be implemented for home monitoring. The NIRS imaging/reading device described herein fulfills these criteria as it is a portable, non-invasive, and contact free device while the leg elevation protocol takes less than 5 minutes to perform. This is considerably shorter than the gold standard ABIs which take on average 10-20 minutes to perform. Second, the leg elevation protocol does not involve extra equipment (e.g., treadmills, occlusion cuffs) that exercise and PORH protocols require. Third, NIRS imaging/reading has good-to-excellent reliability and is a reproducible measurement. Compared to ABIs, which are user dependent, NIRS imaging/reading may be a more objective measurement. Finally, NIRS imaging/reading can give a comprehensive examination of tissue oxygenation within the foot whereas ABIs can only determine perfusion pressure of larger arterioles that provide perfusion the foot.

PAD NIRS imaging/reading with elevation is more effective than common ABIs which can be falsely elevated and do not measure focal perfusion in the lower extremity. This is supported by other work where NIRS imaging/reading detected lateral hypoxemia of the dorsum of the foot both before and after revascularization surgery. As such, NIRS imaging/reading gives a global view of the foot or lower leg which can more precisely identify areas of concern when used in conjunction with a leg raise.

Provocative maneuvers can be used to statistically identify differences in PAD verses control subjects, suggesting potential viability of NIRS for identifying PAD. The disclosed examples build on this as there is a statistically significant difference in ΔStO2, ΔHbO, and ΔHb between PAD and mildly diseased or healthy limbs when elevated, for example, by 45° for 60 seconds. While the data is variable, there were many PAD patients that had a ΔStO2 of at least minus (−)18% which was markedly different than other groups. Further, all PAD patients had an increase in ΔHb, which was not always seen in other groups. Based off this data, NIRS may not be an adequate standalone screen for PAD, but it may be effective in identifying PAD in a fast and precise manner when utilized with a leg elevation protocol. Currently, cut-off values to discriminate PAD from healthy limbs does not seem readily apparent.

Based on the analysis, one unintended finding through the data collection process was that NIRS may be a precise determiner of successful post-surgical outcomes based on two cases.

Tissue oxygen saturation (StO2) is a potentially useful measurement in assessing patient's microcirculatory levels, and, particularly, with assessing the presence and extent of peripheral arterial disease (PAD).

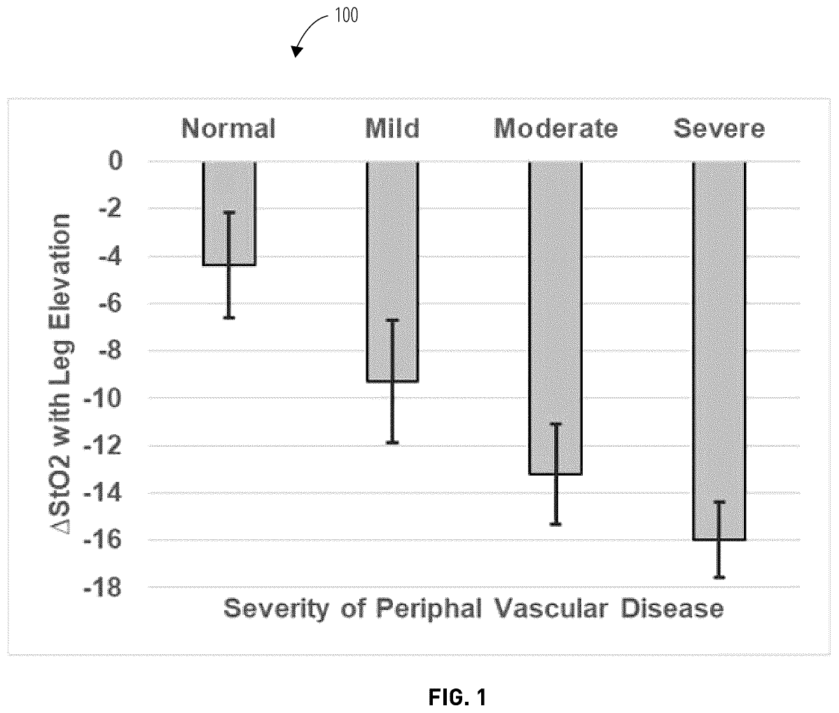

FIG. 1 illustrates an average change in StO2 with provocative maneuver. The provocative maneuver may be considered an elevation of a limb, such as a leg or an arm. Data is represented as means+/−standard error of the mean (SEM): Normal is represented as (n=6), mild (n=5), moderate (n=8), and severe (n=6).

There is no discernable difference in the baseline supine images between all groups. The average value for the mild PAD group was 74+/−7% and the severe PAD group was 76+/−10%. There was a discernable difference between all groups for the delta change from the baseline to the elevation as shown in FIG. 1.

FIG. 2 shows images of a single subject that underwent provocative testing according to disclosed subject matter. The NIRS images from the single subject who underwent the provocative testing before vascular intervention (top images in row labeled Pre-Revasc) and after vascular intervention (Bottom images in row labeled Post-Revasc). Note “Revasc” is an abbreviation for revascularization, which is, for example, a procedure to restore blood flow in block arteries or veins. The image/reading in the upper left corner (Pre-Revasc, Supine) shows different percentages of oxygenation at different points of the subject's foot. Even in the presence of PAD, higher percentages of oxygenation are expected in limbs, such as feet in this case, when in the supine position due to the effect of gravity on the subject's blood. In this example, the NIRS reading in the Pre-Revasc, Supine quadrant indicate percentages of oxygenation at 78% (big toe), 76% and 84% (near small toe). In contrast, when the patient's foot utilizes a provocative maneuver, such as being elevated for a period of time, such as 60 seconds or the like, the percentage of oxygenation is much lower as shown in the Pre-Revasc, Elevated quadrant where the percentages of oxygenation for substantially the same locations on the patient's foot are 51%, 47% and 55%. This illustrates that blood flow is limited to the areas of the patient's foot due to the likely presence of PAD. These lower percentages of oxygenation indicate the presence of or the potential for PAD.

The bottom row of FIG. 2 illustrates the effects of revascularization. In the Post-Revasc, Supine quadrant, the NIRS readings indicate oxygenation percentages of 85%, 79% and 83% for substantially the same locations on the patient's foot as in the Pre-Revasc, Supine reading. More strikingly, the improvement shown in the Post-Revasc, Elevated readings indicates oxygenation percentages of 73%, 70% and 74% for substantially the same locations on the patient's foot as in the Pre-Revasc, Elevated reading.

FIG. 2 illustrates the advantage of utilizing a provocative maneuver in combination with NIRS readings to screen a user for PAD. As a result of the evaluation, a patient may be selected to receive revascularization. And, as a result of the revascularization, oxygenation was preserved in the elevated limb state after revascularization compared to pre-revascularization. A repeat of the NIRS following revascularization can document the adequacy of oxygenation and be used to provide a baseline for future comparisons and determinations of the effectiveness of PAD treatments.

The NIRS readings and indications of percentage oxygenation shown in FIG. 2 are also examples of the images with indications (i.e., the percentage oxygenation) that may be presented on a display/input device (shown in a later example) based on the evaluation of the respective NIRS readings.

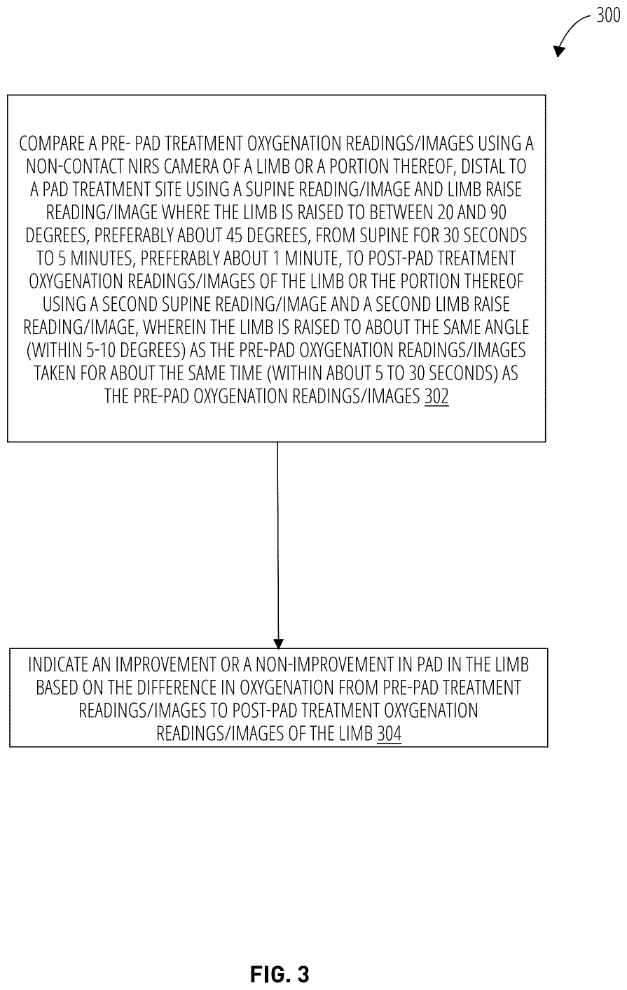

Patients suspected of having PAD were assessed using a provocative leg raising maneuver by elevating the leg at 45 degrees for 60 seconds. Patients were imaged with a non-contact NIRS camera while resting supine and after transient leg elevation. Tissue oxygen saturation (StO2) values were obtained at supine and leg elevation positions. When possible, ABI and TBI were also recorded. FIG. 3 illustrates a routine 300 for determining effectiveness of PAD treatment in accordance with ne embodiment.

In an example, a non-contact NIRS camera may include a processor, memory, and a display device (described in a later example). The memory may be operable to store programming code as a software module. The module may be configured, when executed by the processor, to compare a first oxygenation reading of a limb or a portion thereof of an individual to be evaluated for PAD, who has been diagnosed as having PAD, or who has received a PAD treatment to a second oxygenation reading of the limb or the portion thereof.

In a more detailed example, the routine 300, at block 302, compares pre-PAD treatment oxygenation readings/images obtained by using a non-contact NIRS camera of a limb or a portion thereof, distal to a PAD treatment site using a supine reading/image and limb raise reading/image. For example, the supine reading/image may be obtained with the limb supine against a surface and the limb raising readings/images may be obtained by the non-contact NIRS camera after the individual has raised the limb to between approximately 20 and 90 degrees, preferably about 45 degrees, from supine for approximately 30 seconds to 5 minutes, preferably about 1 minute. The obtained limb raising readings/images may be compared to post-PAD treatment oxygenation readings/images of the limb or the portion thereof using a second supine reading/image and a second limb raise reading/image obtained by the non-contact NIRS camera. The second limb raise reading/image is obtained, substantially like the set of pre-PAD treatment oxygenation reading/image, while or after the limb is raised to approximately the same angle (within 5-10 degrees) as the pre-PAD treatment oxygenation readings/images taken for approximately the same time (within about 5 to 30 seconds) as the pre-PAD treatment oxygenation readings.

The respective post-PAD treatment oxygenation readings and the pre-PAD treatment oxygenation readings may be evaluated according to the PAD severity thresholds described with reference to FIG. 1.

In block 304, the routine 300 indicates an improvement or a non-improvement in PAD in the limb based on a difference in oxygenation from pre-PAD treatment readings or images to post-PAD treatment oxygenation readings or images of the limb. For example, as a result of the comparison, the processor may be operable to indicate on the display device areas of the limb where PAD is suspected based on threshold data as shown in FIG. 1 or FIG. 2, or a combination of both. Alternatively, or additionally, the system may be operable to use the and discussed with reference to FIG. 1 and the earlier examples.

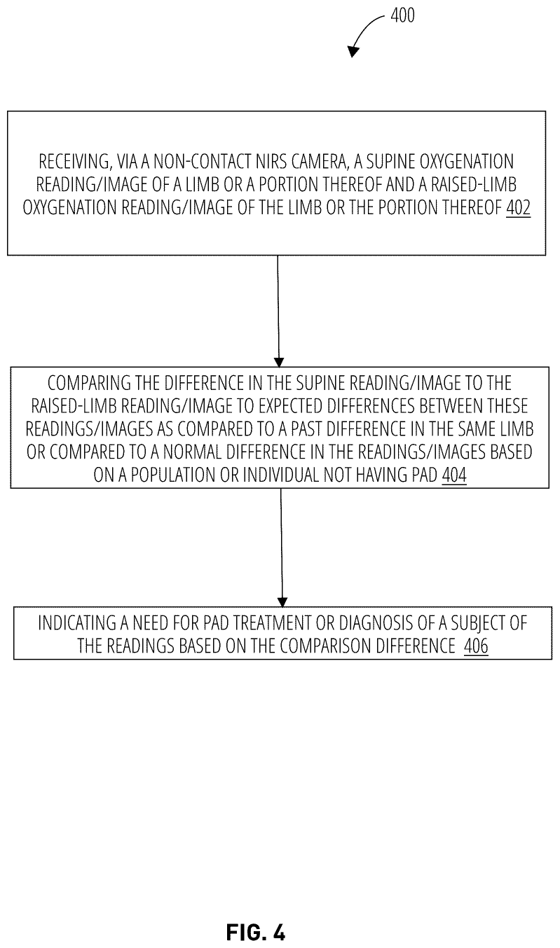

FIG. 4 illustrates a routine 400 for predicting need for peripheral artery disease (PAD) treatment in an individual in accordance with one embodiment.

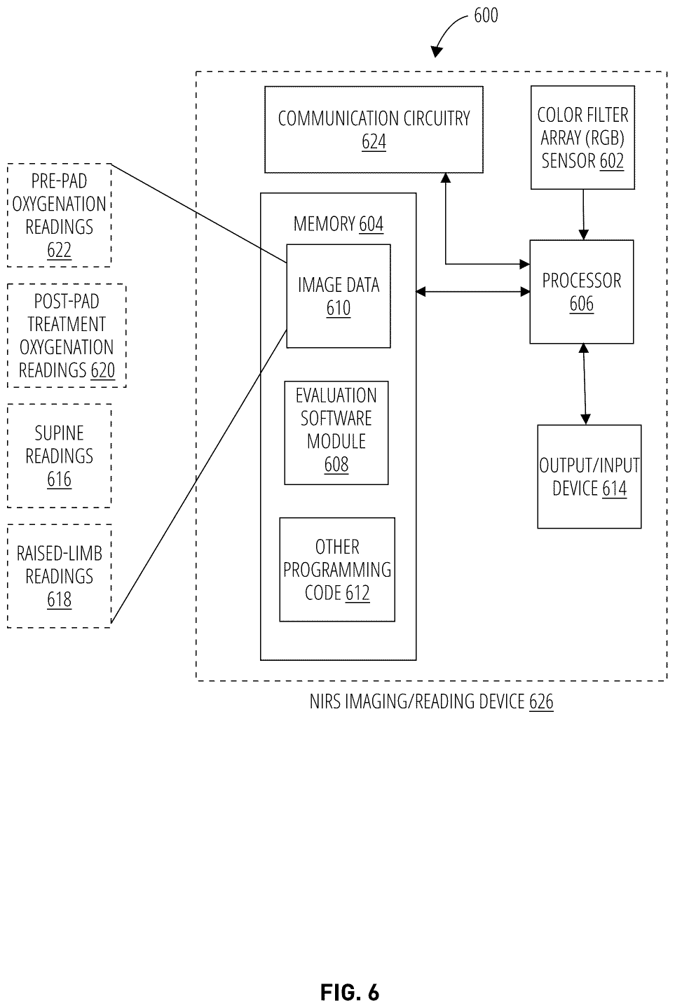

In an example, a non-contact NIRS camera may include a processor, memory, and a display device. The memory may be operable to store programming code as a software module. The module may be configured, when executed by the processor, to execute functions such as those described and needed to produce the results of routine 400. Details of a non-contact NIRS camera are described in more detail with reference to the example of FIG. 6.

As part of routine 400, a non-contact NIRS camera may be operable to take readings (e.g., obtain images) of a limb. For example, in block 402, the processor receives the non-contact NIRS camera obtains a supine oxygenation reading/image of a limb or a portion thereof and a raised-limb oxygenation reading/image of the limb the portion thereof obtained via the non-contact NIRS camera. The raised-limb oxygenation reading/image may be obtained by raising the limb to between 20 and 90 degrees, or preferably about 45 degrees, from supine for 30 seconds to 5 minutes, or preferably about 1 minute.

In block 404, the routine 400 compares the difference in the supine reading/image to the raised limb reading/image to expected differences between these readings as compared to a past difference in the same limb or compared to a normal difference in the readings based on a population or individual not having PAD. The expected difference and the normal difference may be based on evaluations of the severity of PAD as described above with reference to FIG. 1 as well as the other examples.

In block 406, routine 400 indicates need in the individual for PAD treatment or diagnosis based on a result of the difference comparison. For example, as a result of the difference comparison, the processor may be operable to indicate on the display device areas of the limb where PAD is suspected based on difference threshold data as shown in FIG. 2. Alternatively, or additionally, the system may be operable to use the images/readings obtained by an NIRS imaging/reading device and discussed with reference to FIG. 1 and the earlier examples.

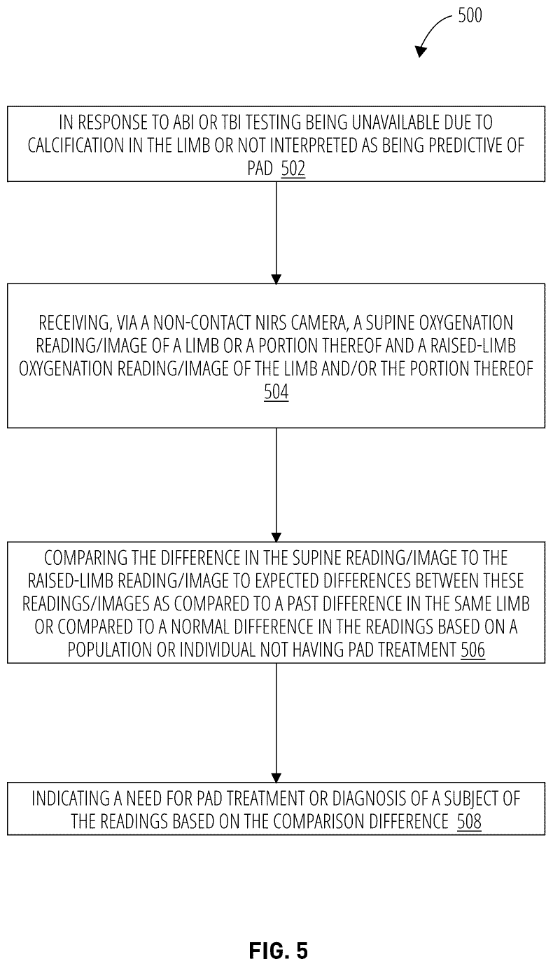

FIG. 5 illustrates a routine 500 for predicting need for peripheral artery disease (PAD) treatment in an individual in accordance with one embodiment.

In an example, a non-contact NIRS camera may include a processor, memory, and a display device. The memory may be operable to store programming code as a software module. The module may be configured, when executed by the processor, to execute functions such as those described and needed to produce the results of routine 500.

As part of routine 500, a non-contact NIRS camera may be operable to take readings (e.g., obtain images) of a limb. For example, in block 502, the routine 500, in response to ABI or TBI testing being unavailable due to calcification in the limb or not interpreted as being predictive of PAD. In block 504, routine 500 receives, via a non-contact NIRS camera, a supine oxygenation reading/image of a limb or a portion thereof and a raised-limb oxygenation reading/image of the limb or the portion thereof. The raised-limb oxygenation reading/image of the limb or the portion thereof is obtained when the limb is raised to between 20 and 90 degrees, preferably about 45 degrees, from supine for 30 seconds to 5 minutes, preferably about 1 minute. In block 506, routine 500 compares the difference in the supine reading/image to the raised limb reading/image to expected differences between these readings as compared to a past difference in the same limb or compared to a normal difference in the readings/images based on a population or individual not having PAD. In block 508, routine 500 indicates need in the individual for PAD treatment or diagnosis based on the difference comparison.

FIG. 6 illustrates an example block diagram of an exemplary system for implementing aspects of the disclosed subject matter.

The NIRS system 600 comprises a color filter array (RGB) sensor 602, a memory 604, a processor 606, communication circuitry 624, and an output/input device 614. The NIRS system 600 may be embodied in a single device such as NIRS imaging/reading device 626, which is also referred to herein as a non-contact NIRS camera. In an example, the NIRS imaging/reading device 626 is a hand-held device usable in an examination room by a healthcare provider without need for a supporting apparatus or structure. Alternatively, the NIRS imaging/reading device 626 may be configured with attachments or connection points to enable the NIRS imaging/reading device 626 to be connected to a supporting apparatus, such as a stationary stand, a moveable trolley, a gantry, gimble, or a combination thereof. In another alternative, the supporting apparatus may have a clamping mechanism or the like that is configurable to hold the NIRS imaging/reading device 626.

In the NIRS system 600, the color filter array (RGB) sensor 602 is coupled to the memory 604. The image data collected by the color filter array (RGB) sensor 602 may be read out to the memory 604. The processor 606 may be operable to access the image data stored in the memory 604, evaluate the image data (interchangeably referred to as “readings”) as discussed herein and generate an indication based on a result of the evaluation.

The color filter array (RGB) sensor 602 may be a red-green-blue (RGB) color image sensor employing a color filter array (CFA) mask in front of an array image sensor thereby providing subsets of pixels sensitive to red, green, or blue light. The color filter array (RGB) sensor 602 is operable to receive infrared light, which is different from other RGB sensors. In most RGB sensors, an infrared filter is employed to block infrared light from impinging on the imaging sensor; however, the image processing utilizes the response of the RGB sensor to infrared light to obtain and generate images or readings of tissue for oxygenation/de-oxygenation analysis. Hence, in at least one example, the color filter array (RGB) sensor 602 either has the infrared blocking filter removed or the sensor does not employ such a blocking filter. In other examples, the color filter array (RGB) sensor 602 may utilize an infrared filter in addition to the RGB filters.

The memory 604 may be configured to store evaluation software module 608, image data 610, other programming code 612, and the like. The other programming code 612 may be In addition, the memory 604 may be configured to store readings by the color filter array (RGB) sensor 802 in the image data 610 as supine readings 616, raised-limb readings 618, Post-PAD treatment oxygenation readings 820, and/or Pre-PAD treatment oxygenation readings 622. All or some of the readings 816-622 may be stored in image data 610. The memory 804 may be a random access memory (RAM), read only memory (ROM), or the like that may be maintained on, for example, any suitable type of memory unit, memory device, memory article, memory medium, storage device, storage article, storage medium and/or storage unit, for example, memory (including non-transitory memory), or the like.

The evaluation software module 608 may be, for example, a module, when executed by the processor 606, configured to compare a first oxygenation reading of a limb or a portion thereof of an individual to be evaluated for PAD or who has been diagnosed as having PAD or who has received a PAD treatment to a second oxygenation reading of the limb or the portion thereof. The evaluation software module 608 may be also operable to perform other functions, such as those functions described with reference to the examples of FIGS. 1-7.

The output/input device 614 may be coupled to the processor 606 and based on an output from the software module 608, the processor 606 may be operable to indicate an improvement or a non-improvement in PAD in the limb based on the difference in oxygenation from pre-PAD treatment readings to post-PAD treatment oxygenation readings of the limb. Alternatively, the processor 606 may be operable to indicate, via the output/input device 616, a need for PAD treatment or diagnosis of a subject of the readings based on the comparison difference. The output/input device 616 may be a display device including a touch screen, a keyboard, keypad, microphone, a speaker, or the like.

The communication circuitry 624 may be one or more transceivers operable to transmit and receive image (reading) data, evaluation results, programming instructions, inputs from remote devices, and the like. The one or more transceivers may be configured to operate according to different wireless or wired communication protocols, such as Bluetooth® including BLE, Wi-Fi, LAN, cellular, or the like.

In an operational example of the NIRS system 600, the NIRS imaging/reading device 626 may be operable to obtain a first oxygenation reading of a limb or a portion thereof via the color filter array (RGB) sensor 602. The color filter array (RGB) sensor 602 of the NIRS imaging/reading device 626 is also operable to obtain a second reading following PAD treatment. The readings may be taken distal to a site of the PAD treatment and where both readings are taken in a raised-limb position, where the limb is raised to between 20 and 90 degrees, preferably about 45 degrees, from supine for 30 seconds to 5 minutes, preferably about 1 minutes.

The evaluation software module 608, when executed by the processor 606, is configured to compare a first oxygenation reading of a limb or a portion thereof taken prior to a PAD treatment to a second oxygenation reading of the limb or the portion thereof. For example, the first reading may be of a limb of an individual that may have been evaluated for PAD, or had been diagnosed as having PAD, while the second reading is taken following the PAD treatment (i.e., post-PAD treatment).

In a further example, the first reading, as processed by the evaluation software module 608 executed by the processor 606, may present a first difference in oxygenation of the limb or the portion thereof in a supine position and the limb or the portion thereof in a raised-limb position. The raised-limb position may be a provocative movement, where the limb is raised to between 20 and 90 degrees, preferably about 45 degrees, from supine for 30 seconds to 5 minutes, preferably about 1 minutes. The second reading may present a second difference in oxygenation of the limb or a portion thereof in a supine position and the limb or the portion thereof in a raised-limb position where the limb is raised to between 20 and 90 degrees, preferably about 45 degrees, from supine for 30 seconds to 5 minutes, preferably about 1 minutes. In this example, the second reading is obtained after the PAD treatment or after a time longer than one week since the first reading.

In this example, the first reading is of the limb or the portion thereof in a supine position, and the second reading is of the limb or the portion thereof in a raised-limb position where the limb is raised to between 20 and 90 degrees, preferably about 45 degrees, from supine for 30 seconds to 5 minutes, preferably about 1 minutes.

The evaluation software module 608 may optionally be further configured to cause the processor to provide a prediction or flag for potential diagnosis of PAD disease, an indication of improvement in oxygenation of the limb or the portion thereof, an indication of non-improvement of the individual's PAD disease, or a combination of predictions, flags, or indications. The processor may provide the prediction or flag to the display/input device 614.

Although shown in a single NIRS imaging/reading device 626, it is envisioned that the NIRS imaging/reading device 626 and the evaluation software module 608 may be configured in separate devices.

A non-contact NIRS camera is beneficial point-of-care tool usable to quantify tissue oxygenation, which is also applicable for the assessment of PAD. Future work may be comparable or superior to ABI for detecting PAD when used in conjunction with a provocative maneuver as it can work with calcinosis.

In some embodiments the methods, system, and devices herein, such as the NIRS system 600, is operable to diagnose PAD. In some embodiments the methods, system, and devices herein, such as the NIRS system 600, is operable to screen for severity of PAD. In some embodiments the methods, system, and devices herein, such as the NIRS system 600, is operable to assess the improvement in PAD following a treatment thereof.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various novel and non-obvious aspects. This method of disclosure, however, is not to be interpreted as reflecting an intention that any claim require more features than are expressly recited in that claim. Rather, novel aspects lie in a combination of fewer than all features of any single foregoing disclosed embodiment.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules, but may be represented in hardware and/or firmware. Moreover, although in some embodiments a module may be separately compiled, in other embodiments a module may represent a subset of instructions of a separately compiled program, and may not have an interface available to other logical program units.

In certain embodiments, software modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

Although described in the illustrative context of certain embodiments and examples, it will be understood by those skilled in the art that the disclosure extends beyond the specifically described embodiments to other alternative embodiments and/or uses and obvious modifications and equivalents. Thus, it is intended that the scope of the claims which follow should not be limited by the particular embodiments described above.

What is claimed is:

1. A method for determining effectiveness of peripheral artery disease (PAD) treatment comprising:
receiving, via a non-contact near-infrared spectroscopy (NIRS) imaging device, pre-PAD treatment oxygenation readings of a limb or a portion thereof of a subject, wherein the pre-PAD treatment oxygenation readings include a first supine reading and a first limb raise reading;
comparing the pre-PAD treatment oxygenation readings to post-PAD treatment oxygenation readings of the limb or the portion thereof, wherein the post-PAD treatment oxygenation readings include a second supine reading and a second limb raise reading; and
indicating an improvement or a non-improvement in PAD in the limb based on a difference in oxygenation from pre-PAD treatment readings to post-PAD treatment oxygenation readings of the limb or portion thereof.

2. The method of claim 1, wherein the pre-PAD treatment oxygenation readings received from the non-contact NIRS camera are readings of a limb or a portion thereof, distal to a PAD treatment site, of a supine reading and limb raise reading.

3. The method of claim 2, wherein the non-contact NIRS camera readings of the limb or the portion thereof show the limb raised to between 20 and 90 degrees, and from supine for a duration of approximately 30 seconds to approximately 5 minutes.

4. The method of claim 2, wherein the limb or the portion thereof is raised to approximately 45 degrees, and held raised from supine for approximately 1 minute.

5. The method of claim 1, further comprising:
receiving the post-PAD treatment oxygenation readings of the limb or the portion thereof of the subject from the non-contact NIRS camera.

6. The method of claim 5, wherein the comparing the pre-PAD treatment oxygenation readings to the post-PAD treatment oxygenation readings, comprises:
determining a difference in the tissue oxygenation of the limb or the portion thereof using supine readings and a limb raise readings of the pre-PAD treatment oxygenation readings of the limb or portion thereof and post-PAD treatment oxygenation readings of the limb or portion thereof.

7. The method of claim 5, further comprises:
leaving the limb or portion thereof supine.

8. The method of claim 5, wherein, when obtaining the post-PAD treatment oxygenation readings of the limb or portion thereof of the person, further comprises:
raising the limb to approximately the same angle from supine as the pre-PAD treatment oxygenation readings; and
taking readings for about the same duration from supine as the pre-PAD treatment oxygenation readings.

9. The method of claim 7, wherein approximately the same angle is within 5-10 degrees of the raised limb used for the pre-PAD treatment oxygenation readings.

10. The method of claim 7, wherein the about same duration from supine for the post-PAD treatment oxygenation readings is within approximately 5 to 30 seconds of the about the same duration from supine as the pre-PAD treatment oxygenation readings.

11. A method for predicting need for peripheral artery disease (PAD) treatment in an individual comprising:
receiving, via a non-contact NIRS camera of a system, a supine oxygenation reading of a limb or a portion thereof and a raised-limb oxygenation reading of the limb or the portion thereof,
comparing a difference in the supine reading to the raised limb reading to expected differences between these readings as compared to a past difference in the same limb or compared to a normal difference in the readings based on a population or individual not having PAD, and
indicating, on an output device a need for PAD treatment or diagnosis of a subject of the readings based on the comparison difference.

12. The method of claim 11, wherein the raised-limb oxygenation reading of the limb or the portion thereof is of the limb raised to between 20 and 90 degrees, and from supine for a duration of approximately 30 seconds to approximately 5 minutes.

13. The method of claim 12, wherein the limb or the portion thereof is raised to approximately 45 degrees, and held raised from supine for approximately 1 minute.

14. A method for predicting need for peripheral artery disease (PAD) treatment in an individual comprising:
in response to ankle-brachial index testing or toe-brachial index testing being unavailable due to calcification in a limb or not interpreted as being predictive of PAD, receiving, via a non-contact NIRS camera, a supine oxygenation reading of a limb or a portion thereof and a raised-limb oxygenation reading of the limb the portion thereof,
comparing a difference in the supine oxygenation reading to the raised-limb oxygenation reading to expected differences between these readings as compared to a past difference in the same limb or compared to a normal difference in the readings based on a population or individual not having PAD, and
indicating a need in the individual for PAD treatment or diagnosis based on the difference comparison.

15. The method of claim 14, wherein the raised-limb oxygenation reading of the limb or the portion thereof is of the limb raised to between 20 and 90 degrees, and from supine for a duration of approximately 30 seconds to approximately 5 minutes.

16. The method of claim 15, wherein the limb or the portion thereof is raised to approximately 45 degrees, and held raised from supine for approximately 1 minute.

17. A system, comprising:
a non-contact near-infrared spectroscopy (NIRS) camera;
memory operable to store programming code;
a processor coupled to the memory, wherein the processor, when executing the programming code, is operable to:
receive, via the sensor, pre-PAD treatment oxygenation readings of a limb or a portion thereof of a subject;
compare the pre-PAD treatment oxygenation readings to post-PAD treatment oxygenation readings of the limb or the portion thereof using a second supine reading and a second limb raise reading; and
indicate an improvement or a non-improvement in PAD in the limb based on a difference in oxygenation from pre-PAD treatment readings to post-PAD treatment oxygenation readings of the limb.

18. The system of claim 17, wherein the pre-PAD treatment oxygenation readings received from the non-contact NIRS camera are readings of the limb or the portion thereof, distal to a PAD treatment site, of a supine reading and limb raise reading.

19. The system of claim 18, wherein the pre-PAD treatment oxygenation readings received from the non-contact NIRS camera are readings of the limb or the portion thereof show the limb raised to between 20 and 90 degrees, and from supine for a duration of approximately 30 seconds to approximately 5 minutes.

20. The system of claim 18, wherein the pre-PAD treatment oxygenation readings received from the non-contact NIRS camera are readings of the limb or the portion thereof raised to approximately 45 degrees, and held raised from supine for approximately 1 minute.
